(19) United States
(12) Patent Application Publication
(10) Pub. No.: US 2025/0345261 A1
(43) Pub. Date: Nov. 13, 2025

(54) FORMULATIONS FOR PREVENTING MOSQUITO BITES

(71) Applicant: Yissum Research Development Company of the Hebrew University of Jerusalem Ltd., Jerusalem (IL)

(72) Inventors: Oded SHOSEYOV, Shoham (IL); Yosef PALTIEL, Rehovot (IL); Jonathan BOHBOT, Rehovot (IL); Daniel VOIGNAC, Tel Aviv (IL); Evyatar SAR-SHALOM, Tel Aviv (IL)

(73) Assignee: Yissum Research Development Company of the Hebrew University of Jerusalem Ltd., Jerusalem (IL)

(21) Appl. No.: 19/130,562

(22) PCT Filed: Nov. 15, 2023
(86) PCT No.: PCT/IL2023/051180

§ 371 Date: May 16, 2025

Related U.S. Application Data
(60) Provisional application No. 63/384,142, filed on Nov. 17, 2022.

(51) Int. Cl. A61K 8/73; A61K 8/04; A61K 8/34; A61K 8/49; A61Q 17/02

(52) U.S. Cl. CPC A61K 8/731; A61K 8/04; A61K 8/345; A61K 8/492; A61Q 17/02; A61K 2800/30

(57) ABSTRACT

The invention contemplates methods and formulations for reducing biting by insects.

### TECHNOLOGICAL FIELD

The technology subject of the invention disclosed herein concerns general purpose formulations as well as skin or topical formulations for preventing mosquito bites.

### BACKGROUND

For many, mosquitos are the most dangerous insects on the planet. Mosquitoes and the diseases they spread have been responsible for killing more people than all world wars combined. Mosquitoes transmitting malaria kill more than 600,000 people and infect more than 200 million every year. Millions of others are debilitated by a host of other mosquito-borne diseases, including filariasis, yellow fever, dengue and encephalitis.

The most effective mosquito chemical repellents contain DEET, picaridin, PMD, or IR3535 insecticide, all of which require direct application onto the skin, and usually have short time and distance effective range requiring frequent applications. Electronic or light attracting devices have been proposed for attracting and eradicating mosquitoes, but these have been found expensive and generally less attractive to consumers.

### General Description

Unlike common belief, mosquitoes do not detect blood. A large part of the mosquito's olfactory system is devoted to sniffing out blood sources by detecting markers that suggest presence of blood. Of the 72 types of odor receptors on the antennae of a female mosquito, at least 27 are tuned to detect chemicals found in perspiration. The female mosquito detects semiochemical substances such as carbon dioxide (CO₂) and 1-octen-3-ol (mushroom alcohol, found in exhaled breath) produced from the host. Mosquitoes prefer certain sweat smells more than others because of the proportions of carbon dioxide, octenol, carboxylic acids and other compounds that make up body odor. The most powerful of the semiochemicals are nonanal and sulcatone or 6-methyl-5-hepten-2-one. Detection of these semiochemicals triggers the female mosquito to settle on the skin and puncture the skin to reach a blood source.

The inventors of the technology disclosed herein have developed formulations that are tailored for application onto a subject's skin region to contain or minimize emission of volatile semiochemicals to the environment. The reduced emissions reduce the effective volume of the semiochemicals in the air and thus a mosquito's ability to detect them and be attracted to their source. Despite the fact that formulations of the invention comprise hydrating agents as well as cellulose-based materials that neither, when used alone, has or exhibits repelling properties, nor is capable of directly eradicating mosquito populations, formulations of the invention have demonstrated superior capabilities in reducing semiochemicals emission and insect bites.

In most general terms, the invention concerns a provision of formulations for application onto a skin (or an exposed skin) region of a subject for reducing or preventing biting insects, such as mosquitoes, from puncturing the skin and/or causing bite-related skin conditions and/or insect-borne diseases.

In a first of its aspects, there is provided a skin formulation comprising a skin-hydrating fluid or material and at least one nanocellulose, e.g., cellulose nanocrystals, CNC, for preventing, reducing, or masking skin-semiochemicals from being detected by a biting insect.

The invention further provides use of such skin formulations in preventing or reducing insect bites.

The invention further provides use of a skin formulation comprising a skin-hydrating fluid or a material and at least one nanocellulose, e.g., cellulose nanocrystals, CNC, for minimizing or diminishing or masking or for preventing skin-semiochemicals emitted from a skin of a subject to sufficiently disperse or spread so as to be detected by a biting insect. In other words, formulations of the invention are used to minimize a concentration of the emitted semiochemicals at the vicinity of the subject or the exposed skin to a concentration that is below a detection level of a biting insect.

As further explained herein, formulations of the invention are suitable for reducing semiochemicals concentration above a surface region of the skin emitting said semiochemicals to below 100 ppm or to sub-ppm levels (to ppb levels). By forming a (dry or semi-dry) coat of the formulation on the skin region, the external temperature of the coated skin region may also be effectively reduced, which further reduces the insect's attraction to the skin.

Formulations of the invention are typically provided in a form that can be used for application directly onto a skin region of a subject. The formulations typically comprise at least one hydrating agent, such as glycerol and at least one nanocellulose, such as cellulose nanocrystals (CNC).

The hydrating agent is selected amongst such materials that are capable of maintaining a degree of skin humidity by any one or more mechanisms of action. The hydrating agent is typically one used in topical cosmetic or pharmaceutical formulations. Non-limiting examples include glycerol, sorbitol, propylene glycol and mixtures thereof.

The nanocellulose is a cellulose-based material selected from nanofibrillar cellulose (NFC or cellulose nanofibrils, CNF), crystalline nanocellulose (CNC) and bacterial nanocellulose (BNC).

In some embodiments, the nanocellulose is CNC.

In some embodiments, the nanocellulose is NFC.

In some embodiments, the nanocellulose is BNC.

As known in the art, CNC is a fibrous material produced from cellulose. The CNC is typically a high-purity single crystal, characterized by having at least 50% crystallinity. In some embodiments, the CNC is monocrystalline. In some embodiments, the CNC, produced as particles (e.g., as a crystalline material) from cellulose of various origins, is selected to be at least about 100 nm in length. In some embodiments, the particles are at most about 1,000 μm in length. In some embodiments, the CNC particles are between about 100 nm and 1,000 μm in length, between about 100 nm and 900 μm in length, between about 100 nm and 600 μm in length, or between about 100 nm and 500 μm in length.

In some embodiments, the CNC particles are between about 100 nm and 1,000 nm in length, between about 100 nm and 900 nm in length, between about 100 nm and 800 nm in length, between about 100 nm and 600 nm in length, between about 100 nm and 500 nm in length, between about 100 nm and 400 nm in length, between about 100 nm and 300 nm in length, or between about 100 nm and 200 nm in length.

The thickness of the CNC material may vary between about 5 nm and 50 nm.

The particles of CNC may be selected to have an aspect ratio (length-to-diameter ratio) of 10 or more. In some embodiments, the aspect ratio is between 60 and 100.

In some embodiments, the CNC is selected to be between about 100 nm and 400 nm in length and between about 5 nm and 30 nm in thickness.

CNC may be used as commercially available or may be prepared according to known methodologies such as the process described in WO 2012/014213 or its equivalent US application, herein incorporated by reference.

In a given formulation, the ratio amounts between the hydrating agent, e.g., glycerol, and the nanocellulose, CNC, may vary. In some embodiments, the ratio hydrating agent: nanocellulose is between 100:1 to 1:100. In some embodiments, the ratio hydrating agent: nanocellulose is 1:20 w/w.

In some embodiments, formulations used according to the invention comprise equal weight amounts of the hydrating agent and the nanocellulose. In other cases, the amount of nanocellulose in the formulation may be twice, three, four, five or more times greater than the amount of the hydrating agent.

In some embodiments, the ratio hydrating agent: nanocellulose is 1:20, or is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18 or 1:19 w/w. In some embodiments, the ratio is in favor of the hydrating agent.

In some embodiments, the aforementioned ratio is between glycerol and CNC.

Formulations of the invention are typically water-based, namely comprising an amount of water that is sufficient to maintain homogenous and stable formulations. In some cases, to permit sufficient evaporation of the water, or a carrier liquid, in addition to or in place of water, other solvents or liquids may be used, e.g., alcohols, glycols and aqueous formulations of same. In some cases, formulations of the invention may consist the skin-hydrating materials, the at least one nanocellulose and water, while in other cases also include at least one additional non-active agents, and/or pharmaceutically or cosmetically acceptable active agents, such as one or more insect repelling agents. For achieving aims of the present technology, the inclusion of a repellent is not required. Where presence of a repellent is nevertheless desired, the insect or mosquito repellent may be selected as known in the art. Generally, the “repellent” or “repelling agent” is a material that inhibits the attraction of an insect such as a mosquito to the target, e.g., a film of the invention or a skin region of a human subject. When mosquito repellents are concerned, these are materials that are used to distance mosquitoes from a predefined area or prevent mosquito biting. The repellents may be synthetic or naturally derived.

The repellents may be selected amongst volatile plant oils such as citronella oil, castor oil, rosemary oil, lemongrass oil, cedar oil, peppermint oil, clove oil, geranium oil and possibly oils from verbena, pennyroyal, lavender, pine, cajuput, cinnamon, basil, thyme, allspice, soybean, oil of lemon eucalyptus (OLE) and garlic; or may be selected amongst synthetic chemicals such as 2-ethyl-3-hexanediol, dichlorodiphenyltrichloroethane (DDT), N,N-diethyl-meta-toluamide (DEET), 1-(1-methylpropoxycarbonyl)-2-(2-hydroxyethyl) piperidine (also known as picaridin, KBR 3023 and icaridin), ethyl butylacetylaminopropionate (IR3535), para-menthane-diol (PMD), 2-undecanone and borneol.

In some embodiments, the formulation may further comprise indole.

In some embodiments, the formulation is free of any repellent or mosquito repelling agent.

Typically, formulations of the invention are not configured nor intended for use solely as repellents, but are rather configured to prevent or reduce effective emission of volatile semiochemicals from the skin region, so as to prevent the mosquito's olfactory system from identifying the presence of the markers and as a result to trigger the mosquito to bite. Without wishing to be bound by any mechanistic description, it is believed that formulations of the invention either prevent emissions of the semiochemicals or dilute their concentrations to below such levels that are detected by the insect's olfactory system or which are sufficient trigger biting. Independent of the mechanism of action, data presented herein demonstrates that skin samples treated with a formulation of the invention, as compared to skin samples treated with either the hydrating agent alone or the nanocellulose alone, demonstrated superior reduction in skin bites, suggesting uniqueness and superiority.

In cases insect repellents are present, a repelling mechanism may also be involved.

As used herein, the term “semiochemicals” refers to chemical substances or mixtures of substances that are released or emitted through a skin or in the breath of the subject, e.g., a human subject, and which attract a biting insect due an interaction with the insect's olfactory system. The semiochemicals may be diverse and include more than one chemical substance. The semiochemicals, which formulations of the invention aim at blocking or interrupting, include any of the volatile chemical substances or mixtures of such chemicals that are released by the subject mainly through the skin, and which the biting insect olfactory system identifies as an indication for a food (blood) source. Without limitation, the semiochemicals may be carbon dioxide, 1-octen-3-ol and other octenols, carboxylic acids, nonanal, sulcatone or 6-methyl-5-hepten-2-one and others, or any combination of volatiles that comprises one or more of the aforementioned. By blocking release of the semiochemicals from the skin to the environment or by preventing their detection by the insects, insect bites may be reduced, or prevented; preventing also a variety of skin conditions and transfer of infectious microorganisms that may be associated with or caused by the biting action. Reducing insect biting may also dramatically reduce human exposure to insect-borne diseases.

Thus, the invention further provides a formulation for use in a method for reducing prevalence of a biting insect (e.g., a mosquito) in a subject, the method comprising forming a coat or a film of a formulation (e.g., water based) comprising a skin-hydrating material, at least one nanocellulose and optionally at least one additive, on a skin of the subject, the coat or film having at least one characteristic sufficient to prevent semiochemicals emitted from the skin to cross the coat or film; or sufficient to reduce the concentration of the semiochemicals from crossing the coat or film to a concentration below that detectable by the biting insect.

Also provided is a method of applying on a skin of a subject a formulation as disclosed herein in an amount (or a thickness) sufficient to form a film capable of reducing prevalence of insect biting.

The film or coat of a formulation of the invention need not be a thick film or coat and there is no need to produce a fully continuous layer. It is sufficient, for the purpose of reducing the amount or concentration of the semiochemicals, to cover a substantial region of an exposed skin. The forming of the film or coat may be achievable by any way, as disclosed herein. As stated herein, the coat or film has at least one characteristic sufficient to prevent semiochemicals emitted from the skin to cross the coat or film; or a characteristic that renders the film or coat sufficient to reduce the concentration of the semiochemicals crossing the coat or film to a concentration below that detectable by a biting insect. The characteristic may be (i) a film/coat thickness of between several microns to several millimeters (e.g., 10 microns to 1 mm), (ii) a skin coverage of at least 50% (or 60, or 70, or 80, or 90, or 100%) of the exposed skin surface, and (iii) optional inclusion of an insect repellent.

In some embodiments, the characteristic is a film/coat thickness.

A typical biting insect such as a mosquito is capable of detecting semiochemicals from several meters and is capable of detecting semiochemicals concentrations ranging from mid-range ppb to mid-range ppm levels. Thus, reducing the concentration of such materials emitted from a skin region to levels below 100 ppm or even to below 100 ppb reduces a mosquito's olfactory system ability to detect the presence of these materials and as a result to trigger the mosquito to bite.

The biting insect may be any of the known insects, mites, and ticks which are equipped with a proboscis that is structured to puncture the skin to inject saliva for the purpose of digesting a tissue and aid in the feeding process. Typically, the insect is a mosquito.

The mosquito may be any of the known mosquito species derived from the mosquito genus Aedeomyia, Aedes, Anopheles, Armigeres, Ayurakitia, Borachinda, Coquillettidia, Culex, Culiseta, Deinocerites, Eretmapodites, Ficalbia, Galindomyia, Haemagogus, Heizmannia, Hodgesia, Isostomyia, Johnbelkinia, Kimia, Limatus, Lutzia, Malaya, Mansonia, Maorigoeldia, Mimomyia, Onirion, Opifex, Orthopodomyia, Psorophora, Runchomyia, Sabethes, Shannoniana, Topomyia, Toxorhynchites, Trichoprosopon, Tripteroides, Udaya, Uranotaenia, Verrallina and/or Wyeomyia.

In some embodiments, the mosquito is selected from the genus Aedes.

In some embodiments, the mosquito is selected from Aedes australis, Aedes aboriginis, Aedes aegypti, Aedes africanus, Aedes albolineatus, Aedes alboniveus, Aedes albopictus, Aedes albolineatus, Aedes alboscutellatus, Aedes aloponotum, Aedes amesii, Aedes annulipes, Aedes arboricola, Aedes argenteoventralis, Aedes atlanticus, Aedes atropalpus, Aedes aurifer, Aedes aurimargo, Aedes aurotaeniatus, Aedes axitiosus, Aedes bahamensis, Aedes barraudi, Aedes bekkui, Aedes bicristatus, Aedes bimaculatus, Aedes brelandi, Aedes brevitibia, Aedes burger, Aedes cacozelus, Aedes camptorhynchus, Aedes canadensis, Aedes cantans, Aedes cantator, Aedes caspius, Aedes cataphylla, Aedes cavaticus, Aedes cinereus, Aedes clivis, Aedes communis, Aedes cordellieri, Aedes coulangesi, Aedes cretinus, Aedes dasyorrhus, Aedes deserticola, Aedes desmotes, Aedes domesticus, Aedes dupreei, Aedes eldridgei, Aedes epactius, Aedes esoensis, Aedes fulvus, Aedes furcifer, Aedes futunae, Aedes Ganapathi, Aedes geminus, Aedes gombakensis, Aedes grassei, Aedes grossbecki, Aedes harinasutai, Aedes helenae, Aedes hensilli, Aedes hesperonotius, Aedes hoogstraali, Aedes horotoi, Aedes imprimens, Aedes inermis, Aedes infirmatus, Aedes intrudens, Aedes japonicus, Aedes kochi, Aedes kompi, Aedes koreicus, Aedes lineatopennis, Aedes luteocephalus, Aedes madagascarensis, Aedes malayensis, Aedes marshallii, Aedes masculinus, Aedes mediolineatus, Aedes mediovittatus, Aedes mefouensis, Aedes melanimon, Aedes meronephada, Aedes michaelikati, Aedes mitchellae, Aedes mohani, Aedes monticola, Aedes muelleri, Aedes nevadensis, Aedes ngong, Aedes niphadopsis, Aedes niveus, Aedes notoscriptus, Aedes nummatus, Aedes ostentation, Aedes palpalis, Aedes pembaensis, Aedes pexus, Aedes polynesiensis, Aedes pseudoniveus, Aedes pseudonummatus, Aedes pulchritarsis, Aedes pullatus, Aedes pulverulentus, Aedes punctodes, Aedes punctor, Aedes purpureipes, Aedes purpureifemur, Aedes rempeli, Aedes rusticus, Aedes scapularis, Aedes schizopinax, Aedes scutellaris, Aedes sollicitans, Aedes spilotus, Aedes squamiger, Aedes stricklandi, Aedes sylvaticus, Aedes taeniorhynchus, Aedes taylori, Aedes thelcter, Aedes thibaulti, Aedes thomsoni, Aedes tiptoni, Aedes togoi, Aedes tormentor, Aedes tortilis, Aedes turneri, Aedes varipalpus, Aedes ventrovittis, Aedes vexans, Aedes vigilax, Aedes vittatus, Aedes washinoi, Aedes wauensis and Aedes zoosophus.

In some embodiments, the mosquito is Aedes aegypti.

The invention further provides a method for controlling insect (e.g., mosquito) biting activity by the application of an effective amount of a formulation of the invention to an area of a subject's skin where such insect control is desired. The application of the formulation may or may not be to the complete skin of the subject. Depending on the particular need and the degree of exposure of one skin region relative to another, the application may be to only such regions that are not dressed or covered, but rather are exposed or more exposed to insects. Such exposed skin regions may be hands, face, legs, neck, and others. The application onto a skin region, e.g., exposed skin region, may be achieved by any means known and available for application of creams, foams, gels, lotions, sprays and ointments, as topical formulations. For ease of application and consumer convenience, formulations of the invention, comprising the hydrating agent and the nanocellulose, may be combined with other cosmetic formulations or ingredients in order to provide a multipurpose formulation. Thus, formulations may be formed into creams, lotions, ointments, sprays etc.

For ease of application, particularly over large skin regions, formulations of the invention may be provided in a form of a sprayable formulation or a roll-on formulation or may be provided absorbed in a wipe or a brush or an applicator of any sort. Thus, the invention further provides an applicator, e.g., a roll-on, for applying a skin formulation onto a skin region of a subject.

By preventing or minimizing or reducing mosquito or insect biting, a myriad of skin conditions and other diseases may be prevented. The skin conditions may vary from itchy bumps that form on the skin to inflamed and severe allergic skin reactions. Mosquito bites can also cause severe illnesses if they carry certain bacteria, viruses or parasites. Such mosquito-borne diseases may include malaria, dengue, West Nile virus, chikungunya, yellow fever, dirofilariasis, filariasis, tularemia, Japanese encephalitis, Saint Louis encephalitis, Western equine encephalitis, Eastern equine encephalitis, Venezuelan equine encephalitis, Ross River fever, Barmah Forest fever, La Crosse encephalitis, Zika fever and others. Thus, in preventing insect bites, formulations of the invention further prevent skin conditions and other insect-borne diseases.

The invention thus further provides a method of preventing or reducing skin biting by at least one biting insect, the method comprising applying onto a region of the skin an effective amount of a formulation comprising at least one hydrating agent and at least one nanocellulose.

The effective amount of a formulation according to the invention is an amount that suffices to coat a skin region with a film of between several microns to several millimeters in thickness. Coating may be continuous or partial as long as a substantial percentage of exposed skin is coated. Provided that formulations of the invention comprise a homogeneous mixture of the hydrating agent and the nanocellulose, such a film may provide sufficient masking capabilities.

As stated herein, the invention contemplates insect, e.g., mosquito, repellent formulations comprising at least one nanocellulose, as defined and selected, at least one skin hydrating agent and optionally at least one insect repellent.

Also provided is an insect, e.g., mosquito, repellent formulation, the formulation comprising at least one nanocellulose, as defined and selected, at least one plasticizer and at least one insect repellent.

The invention further provides an insect repellent formulation, the formulation comprising at least one nanocellulose, as defined and selected, glycerol and at least one insect repellent.

Further provided is a film forming repellent formulation, the formulation comprising at least one nanocellulose, as defined and selected, at least one plasticizer and at least one insect repellent, wherein the formulation is configured for direct application onto a substrate to thereby form an insect repellent film.

The invention further provides an object or an article having a surface region provided with a repellent film according to the invention.

The general-purpose formulations of the invention, namely those intended for application on surface region of objects or articles, comprise a nanocellulose as defined and selected and at least one plasticizer which may be any such material known in the art, which when added to the CNC to form a film, provides a film with reduced brittleness, and improved processability and flexibility, thereby avoiding cracking, e.g., during application and drying.

Non-limiting examples of plasticizers include glycerol, water, polyethylene glycol, propylene glycol, monoacetin, triacetin, triethyl citrate, sorbitol, 1,3-butanediol, diethylene glycol, castor oil, and combinations thereof. In some embodiments, the plasticizer is glycerol.

Films formed of formulations of the invention are typically continuous films, demonstrating few or no cracking, thus providing an effective coating. The films may be formed on a surface of any substrate by applying a thin film of a formulation and allowing the formulation to dry or form into a solid film. Release of the repellent from the film and into the environment may be spontaneous or may be controlled or tailored to meet a certain release profile.

Objected provided with repellent films of the invention may include bad nets, window screens, repellent tags, textiles, and any other object, device or article.

The invention further provides a kit or commercial package comprising a formulation of the invention and instructions of use.

In some embodiments, the kit may also include an applicator, which may or may not already contain the formulation. In some cases, the formulation and the applicator are provided separately within the same kit and are adapted to be used in combination. For example, the applicator may be a brush, a roll-on, a cloth, a spray or any other tool that can be used to apply the formulation onto the skin of the subject.

In some cases, the formulation may be contained in the applicator. Such an applicator may be a roll-on or a cloth which is saturated or is wet with the formulation.

In some cases, the formulation may be provided dry, e.g., as a powder, and the used may be instructed to add a certain amount of water to solubilize or disperse its components, as disclosed herein.

The invention further provides an applicator unit containing a formulation of the invention and having an end for delivering said formulation onto a skin region by contacting the skin region with said end or by spraying said formulation. The applicator may be a roll-on or a spray unit.

The invention further provides:

A formulation for direct application onto a skin region of a subject for reducing or preventing insects from biting the skin region and/or for reducing or preventing bite-related skin condition and/or for reducing or preventing transmittal of an insect-borne disease, the formulation comprising a skin-hydrating fluid and at least one nanocellulose.

In some embodiments of formulations according to the invention, the reducing or preventing is achievable by minimizing, diminishing or preventing skin-semiochemicals emitted from the skin region to sufficiently disperse or spread so as to be detected by the insect.

In some embodiments of formulations according to the invention, the reducing or preventing is achievable by minimizing or reducing concentration of skin-semiochemicals emitted from the skin region to below 100 ppm or to below 100 ppb level and/or by reducing external temperature of the skin region having the formulation applied thereon.

In some embodiments of formulations according to the invention, the reducing or preventing is by forming a film of the formulation having a thickness of between 10 microns and 1 mm.

In some embodiments of formulations according to the invention, the hydrating agent is selected amongst materials capable of maintaining a degree of skin humidity.

In some embodiments of formulations according to the invention, the hydrating agent is glycerol, sorbitol, propylene glycol or mixtures thereof.

In some embodiments of formulations according to the invention, The formulation according to claim 6, wherein the hydrating agent is glycerol.

In some embodiments of formulations according to the invention, the nanocellulose is selected from nanofibrilar cellulose (NFC), crystalline nanocellulose (CNC) and bacterial nanocellulose (BNC).

In some embodiments of formulations according to the invention, the nanocellulose is CNC.

In some embodiments of formulations according to the invention, the hydrating agent is or comprises glycerol and wherein the nanocellulose is CNC.

In some embodiments of formulations according to the invention, a weight ratio hydrating agent: CNC is 100:1 to 1:100.

In some embodiments of formulations according to the invention, the ratio hydrating agent: CNC is 1:20.

In some embodiments of formulations according to the invention, the formulation being free of an insect repellent.

In some embodiments of formulations according to the invention, the formulation comprising at least one insect repellent.

In some embodiments of formulations according to the invention, the insect repellent is selected amongst citronella oil, castor oil, rosemary oil, lemongrass oil, cedar oil, peppermint oil, clove oil, geranium oil, oil of verbena, oil of pennyroyal, oil of lavender, oil of pine, oil of cajuput, oil of cinnamon, oil of basil, oil of thyme, oil of allspice, soybean oil, oil of lemon eucalyptus (OLE), oil of garlic, 2-ethyl-3-hexanediol, dichlorodiphenyltrichloroethane (DDT), N,N-diethyl-meta-toluamide (DEET), 1-(1-methylpropoxycarbonyl)-2-(2-hydroxyethyl) piperidine, ethyl butylacetylamino propionate (IR3535), para-menthane-diol (PMD), 2-undecanone and borneol.

In some embodiments of formulations according to the invention, the formulation comprising indole.

In some embodiments of formulations according to the invention, the formulation comprising CNC, glycerol and indole.

In some embodiments of formulations according to the invention, the insect is a mosquito.

In some embodiments of formulations according to the invention, the skin semiochemicals are volatile chemical substances or a mixture of substances released by the subject through the skin, and which the biting insect olfactory system identifies as an indication for a food source.

In some embodiments of formulations according to the invention, the semiochemicals are one or more agent selected from carbon dioxide, octenols, 1-octen-3-ol, carboxylic acids, nonanal, 6-methyl-5-hepten-2-one and combination comprising one or more of the aforementioned.

The invention further provides a formulation for minimizing, diminishing or preventing skin-semiochemicals emitted from the skin region to disperse or spread so as to be detected by a mosquito, the formulation being adapted for direct application onto an exposed skin region of a subject for forming a film of said formulation, thereby reducing or preventing mosquitos from biting the skin region, the formulation comprising glycerol, CNC and optionally indole.

In some embodiments of formulations according to the invention, the formulation is for maintaining a concentration of the skin-semiochemicals above the film to be below 100 ppm.

In some embodiments of formulations according to the invention, the formulation comprising indole.

Also provided is a method for preventing or reducing insect biting, the method comprises applying an effective amount of a formulation according to any embodiment or aspect of the invention to an exposed area of a skin of a subject and forming a film or a coat of said formulation on said area of the skin.

In some embodiments of methods according to the invention, the film or coat having at least one characteristic selected to prevent semiochemicals emitted from the skin to cross the film or coat; or selected to reduce the concentration of the semiochemicals crossing the film or coat to a concentration below a concentration detectable by the insect.

In some embodiments of methods according to the invention, the characteristic is a film/coat thickness, a skin coverage, and the inclusion of a skin repellent.

In some embodiments of method according to the invention, the characteristic is a film/coat thickness.

In some embodiments of methods according to the invention, the film/coat thickness is between 10 microns to 1 mm.

In some embodiments of method according to the invention, the concentration of the semiochemicals below the concentration detectable by the insect is below 100 ppm or is below 100 ppb.

In some embodiments of methods and formulations according to the invention, the biting insect is a mosquito.

In some embodiments of methods and formulations according to the invention, the mosquito is selected from a mosquito species derived from the mosquito genus Aedeomyia, Aedes, Anopheles, Armigeres, Ayurakitia, Borachinda, Coquillettidia, Culex, Culiseta, Deinocerites, Eretmapodites, Ficalbia, Galindomyia, Haemagogus, Heizmannia, Hodgesia, Isostomyia, Johnbelkinia, Kimia, Limatus, Lutzia, Malaya, Mansonia, Maorigoeldia, Mimomyia, Onirion, Opifex, Orthopodomyia, Psorophora, Runchomyia, Sabethes, Shannoniana, Topomyia, Toxorhynchites, Trichoprosopon, Tripteroides, Udaya, Uranotaenia, Verrallina and/or Wyeomyia

In some embodiments of methods and formulations according to the invention, the mosquito is selected from the genus Aedes.

In some embodiments of methods and formulations according to the invention, the insect is a mosquito that is Aedes aegypti.

In some embodiments of methods and formulations according to the invention, the methods or formulations are for preventing or minimizing or reducing a mosquito-borne disease selected from malaria, dengue, West Nile virus, chikungunya, yellow fever, dirofilariasis, filariasis, tularemia, Japanese encephalitis, Saint Louis encephalitis, Western equine encephalitis, Eastern equine encephalitis, Venezuelan equine encephalitis, Ross River fever, Barmah Forest fever, La Crosse encephalitis, Zika fever.

In some embodiments of methods and formulations according to the invention, wherein the formulation is in a form of a cosmetic formulation, or as a multipurpose formulation.

In some embodiments of methods and formulations according to the invention, wherein the formulation is in a form of a cream, a lotion, an ointment, or a spray.

In some embodiments of methods and formulations according to the invention, wherein said applying comprising brushing, spraying, wiping, dipping, smearing, oiling, application of a cream, or application by a roll-on.

Also provided is a kit optionally comprising a formulation according to any embodiment or aspect of the invention or a formulation to be used with any method of the invention; and instructions of use.

In some embodiments of kits according to the invention, the kit comprises a skin applicator comprising the formulation.

In some embodiments of kits according to the invention, the skin applicator is a roll-on or a spray.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1: Percent blood fed females in cage with CNC on hand and without CNC on hand). CNC appears to significantly reduce the blood feeding, (P<0.5).

FIG. 2: Eggs laid after Hemotek feeding on A. Aegypti for 1 h through four types of membranes. A collagen membrane (top row), a collagen membrane coated with CNC with 5 wt % glycerol (second row), a stretched Bemis Parafilm (third row), CNC glycerol and 1 mM Indole (bottom row).

FIG. 3: Egg corresponding pixel density average for Hemotek testing on A. Aegypti.

FIG. 4: Eggs laid after Hemotek feeding on A. Aegypti for 1 h through three types of membranes. A collagen membrane (top row), a collagen membrane coated with CNC with 5 wt % glycerol (second row), a stretched Bemis Parafilm (third row).

FIG. 5: Egg corresponding pixel density average for Hemotek testing on A. Aegypti. On average, CNC allowed a 48% reduction in egg laying but this result is not statistically significant.

FIGS. 6A-B: Set-up used for determining VOC permeation through a film according to some embodiments of the invention (B) and results of the measurements (A).

### DETAILED DESCRIPTION OF EMBODIMENTS

### Experimental

### Mosquito Culture

Aedes aegypti

The Aedes aegypti were grown as per Bohbot's et al. methodology subjected to 12 h daylight and 12 h dark environment cycles while being fed with 10 w/w % sucrose solution in water through a cotton ball. Dried A. Aegypti eggs were hatched in water and fed with crab food through their larval phase in the same environmental conditions as the mature mosquitoes. Pupae were collected and placed in developmental cages to reach maturity with sucrose feed changed every 48 h. Mature mosquitoes were transferred to experimental cages and split according to the needs of each treatment. Table 1 outlines the population in each cage for the artificial feeding experiments of A. Aegypti

TABLE 1
Cage population for Hemotek testing on A. Aegypti

| Cage Reference | Females | Males |
|---|---|---|
| Control 1 | 16 | 18 |
| Control 2 | 8 | 8 |
| Control 3 | 15 | 5 |
| CNC 1 | 11 | 9 |
| CNC 2 | 14 | 7 |
| CNC 3 | 14 | 18 |
| CNC + 1 mM Indole 1 | 12 | 17 |
| CNC + 1 mM Indole 2 | 24 | 4 |
| CNC + 1 mM Indole 3 | 17 | 5 |
| Parafilm 1 | 11 | 5 |
| Parafilm 2 | 14 | 9 |
| Parafilm 3 | 10 | 7 |

For the in-vivo experiments on human hand, the mosquitoes were frozen immediately after the experiment allowing monitoring of blood swelling in females thus improving the count. For the artificial membrane (Hemotek) experiments, the mosquitoes were let to feed for 1 h in the lab environment (20 to 23° C. and a relative humidity between 40 and 50%) and immediately placed back in the incubators with a 10 wt % sucrose solution replaced every 24 h to feed on.

### Anopheles Gambiae

The Anopheles Gambiae were grown as per Papathanos' et al. methodology. All cages were prepared with 60 females and 40 males. The sex separation and cage preparation were performed when the insects were at a pupae stage making it much easier to split.

### CNC Preparation

In a preliminary stage, pure CNC (2% wt.) in water supplied by Melodea, Israel was used, titrated with Sodium Hydroxide (NaOH) up to pH 5.5, the average skin pH. However, this formulation cracked on the skin upon drying along the cracks of the skin. The problem was mitigated by adding 0.1 vol % glycerol as a plasticizer to the solution to reach 5 wt % of the dry formulation.

### CNC Indole Preparation

To assess the synergistic effect of CNC as a passive barrier together with an active repellent, indole was dispersed in the CNC glycerol solution. 1 mM of indole was added as a dry powder in the previously described solution. Ultrasonication was used to disperse in the Indole in a QSONICA sonicator (500 Watt; 20 kHz, 1 sec on; 1 sec off frequency) for 5 min at 50% amplitude. Indole was purchased from Sigma-Aldrich. Air bubbles were removed by centrifugation for 3 min at 5,000 rcf.

### Human Hand Testing Apparatus

A 3D printed ring made of two interlocking parts was clipped on a disposable nitril glove, below and above the glove and the inner section of the glove was cut out to reveal a constant and consistent testing area. Mosquitoes were starved one hour prior to experimenting. Experiments were conducted between 9 and 12 am as this is a known window of high activity for the A. Aegypti. at a temperature kept between 20° C. to 23° C. and a relative humidity between 40 and 50%. Lighting and shading were kept constant.

Subject kept left hand immobile in the cage for 10 min in each treatment. Said subject had no adverse reaction to the biting hence, swollen spots were not a valid data point. A camera placed above a transparent face of the cage was used to monitor number of visits. The total exposed skin area was 21.23 cm² not accounting for the curvature of the hand below.

Mosquitoes were cooled to allow counting of blood fed as observed by red body swelling in females.

Data Analysis was done by monitoring the videos for each sampling and counting the number of visits as well as counting the visibly blood fed insects by freezing the cages immediately after the 10 min of exposure.

### Artificial Membrane (Hemotek)

Unlike the human hand experiment, the artificial membrane was left for 1 hour feeding in slightly larger cages. This may have allowed the insects more time to assess less obvious cues such as the heat from the Hemotek.

The Hemotek was filled with 1.5 ml of cow blood obtained from the local slaughterhouse. The collagen membranes supplied by Hemotel were always used, as indicated by the supplier. When CNC and CNC-Indole were applied on the membranes, the membranes were already placed on the feeders and left to dry in open air. Parafilm (Bemis) controls were prepared by stretching Parafilm on top of the collagen membrane to obtain a comparative for the behavior of a dense polymer on top of the collagen membrane. For the A. Aegypti, 2% ATP was added to the blood as a phagostimulant.

Egg collection was done by placing a Whatman filter paper on the surface of a water cup that the eggs laid on the surface could be collected simply by lifting the filter paper. The timing of the oviposition cup varied with the species. For the A. Aegypti it was placed 48 h after blood feeding and only 24 h for the An. Gambiae.

### Results and Discussion

### CNC-Glycerol on Human Hand

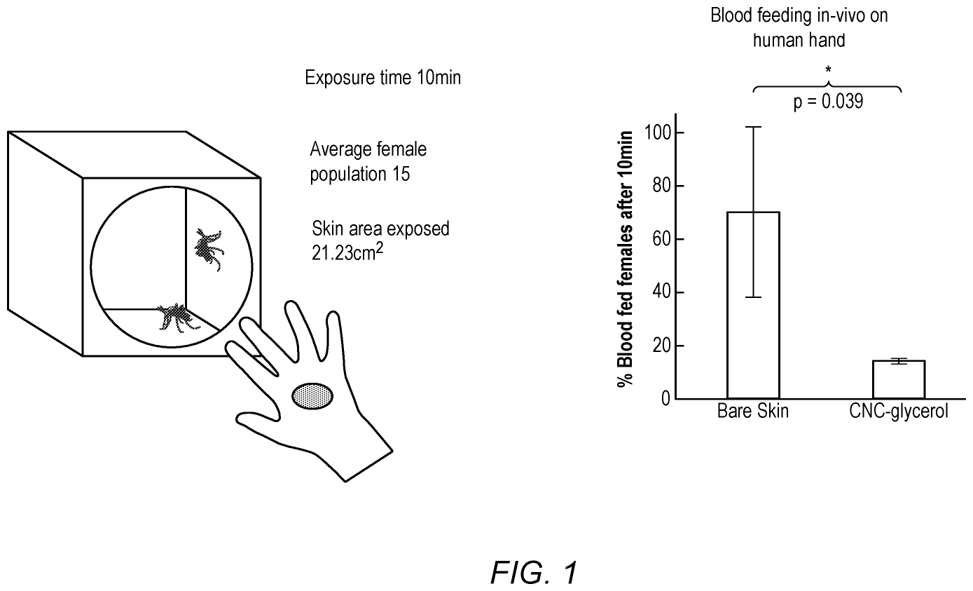

Initial testing was designed as a direct analogy to Castilho et al.'s initial approach¹ and as the most straightforward way to evaluate CNC's effect. The author's hand was placed in a cage with an average of 15 females (Table 1) for 10 min. A constant area of the skin was exposed to the mosquitoes, with and without CNC, on the skin as described in the experimental section. The first conclusion was that CNC (ph 5.5, 2 wt. % in water) did not dry ideally on the skin's surface as its brittleness caused it to crack along the lines of the skin. To alleviate this issue 0.1 wt. % of glycerol was added to the CNC aqueous solution. The CNC-glycerol was applied on the skin as a gel and let dry for a few minutes. For both treatments (with and without CNC-Glycerol) a new set of mosquitoes were used and three cages per treatment were performed. The CNC-glycerol showed a significant and dramatic reduction of blood-fed mosquitoes with 71±31% blood-fed females in nominal conditions after 10 minutes and only 14±1% blood-fed females when CNC-glycerol was applied on skin (FIG. 1) (from this point in writing CNC when referred to as the investigated barrier refers to CNC-glycerol).

This result was encouraging enough to pursue the investigation. The observation of the video monitoring of each experiment revealed that for the CNC coated hands, very few mosquitoes landed on the hand suggesting that the CNC offers a chemical barrier. Moreover, the few females that did land on CNC coated hands systematically blood-fed suggesting that the physical barrier hypothesis was not confirmed. This may be due to uneven spread of the CNC-Glycerol which could be improved with cosmetic formulation. But most probably the CNC-Glycerol does not fully dry on the skin and the complex probing of the mosquitoes' proboscis allows to penetrate through by agitating the molecules and creating a path through to the skin. To further investigate the effect, a more controlled method was attempted, and an artificial feeding system was selected to feed mosquitoes over a longer period.

### Hemotek Artificial Feeding on A. Aegypti

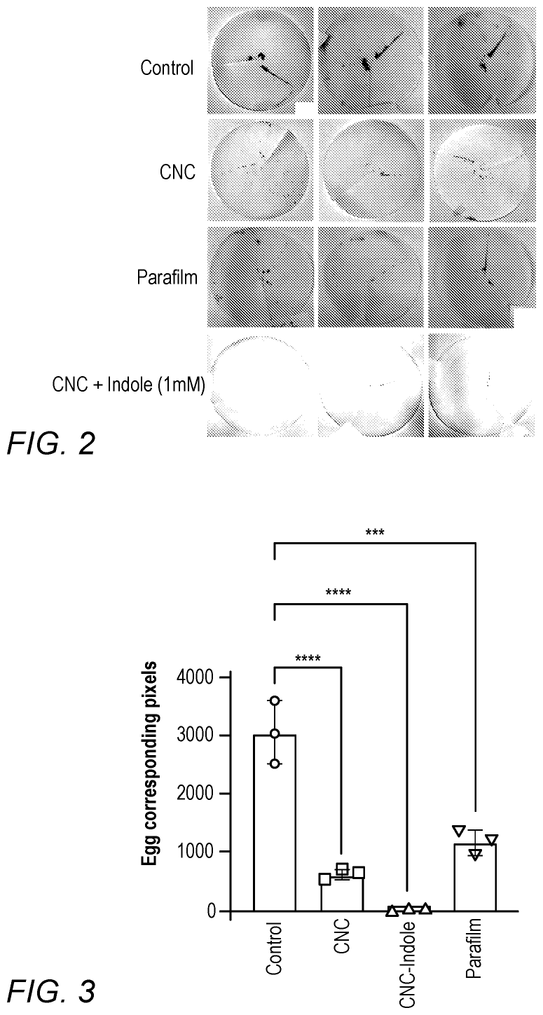

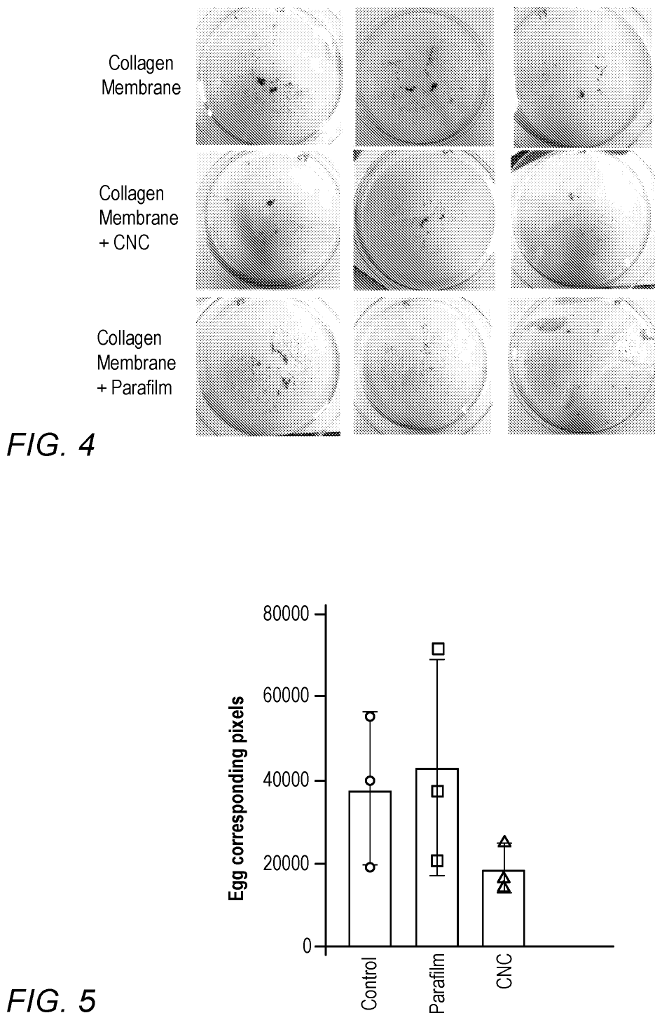

The Hemotek system was used to remove the bias of the human subject and allow a control of more parameters in each cage. All three cages in each treatment were fed in parallel and left to feed for an hour for A. Aegypti. The filter papers on which eggs were collected were captured with the same camera and same lighting to process the results with simple image processing tools. Indeed, the difference in mass between cages was too subtle to accurately measure whereas the dark color of the eggs allowed easy optical counting. The number of eggs was too high for manual counting and the contrast between the eggs and other features in the image enabled to identify the greyscale threshold corresponding to eggs in the image. The threshold was determined using the image processing toolbox in Matlab and the number of pixels on the greyscale below the determined threshold was summed using FIJI image analysis software and the histogram tool. The sums were averaged and compared for each treatment yielding the significant results in FIG. 2.

The reduction in eggs between the control and the CNC was dramatic and confirmed a barrier effect. The parafilm was used as a comparative dense polymer coated on the skin. An added parafilm layer did significantly reduce the number of eggs laid, as is to be expected from an added layer, making it more complex for mosquitos to penetrate through, though not as much as CNC-glycerol. Parafilm is also petroleum derived and not produced in a way that is compatible with skin application. The CNC-Indole coating led to the most effective egg reduction effect, with one cage showing no egg laying at all. This further suggests that CNC is a non-chemical barrier acting as a camouflage to most general cues sought by mosquitoes.

Although the excellent heat conductivity of CNC may play a role in fast dissipation and the transparency and LC behavior may also affect the optical perception of the membrane. The addition of Indole as an active repellent assessed and confirmed the potential synergistic effect towards a total protection solution. CNC-Indole was much better at egg reduction with down to 99.4% reduction in eggs. It combines both the chemical camouflage provided by the CNC with the active chemical repellent that is Indole. The low molarity of the Indole (1 mM) minimizes the discomfort from the Indole smell.

CNC appears to be an effective barrier method to mosquito egg laying. Egg laying is directly correlated to biting and feeding effectiveness. Thus, combining the observations in the human hand experiment and the Hemotek experiment suggests that CNC could be an effective barrier to mosquito biting. While not being a perfect barrier, the combination of CNC with active repellents would be an excellent route to pursue. The intrinsic properties of the CNC allow efficient dispersion of the Indole and could be an ideal method for aqueous based dispersion and slow-release mechanism for active repellents that in general require organic solvents to be effective.

Hemotek Artificial Feeding on An. Gambiae

An. Gambiae were chosen for the great threat they pose as vectors of Malaria and for their genetic distance to A. Aegypti. The latter could likely assess the effectiveness of CNC as a barrier across 150-200 million years of mosquito evolution. In this experiment the sample populations chosen were much larger with 60 females and 40 males in each cage. The cages were left to feed for two hours and the egg collection was adapted to egg laying cycle of the species. The egg density was assessed in the same manner as described earlier for the A. Aegypti. The camera and lighting were different than the A. Aegypti sampling but kept the same for all An. Gambiae explaining the difference in pixel density between the species.

The image analysis summarized in FIG. 5 shows a 48% average reduction in eggs with CNC as compared to the control membranes. However, the differences between the treatments do not appear as statistically significant.

### VOC Permeation Through the Film

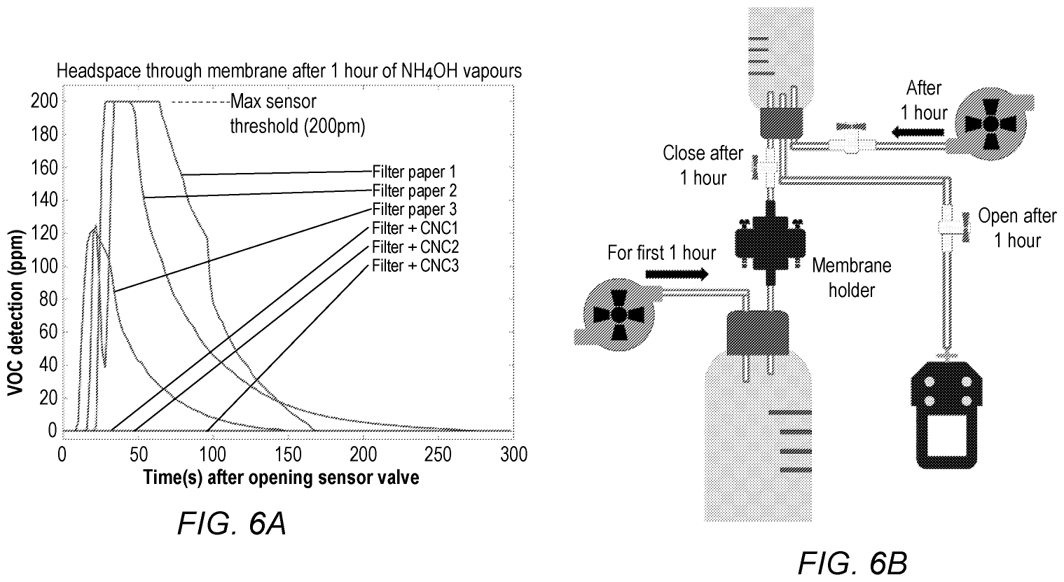

To assess the chemical barrier ability, a headspace experiment was designed by adapting volatile usage and flow from Smallegange et al. The designed setup is illustrated in FIG. 6B. In this experiment, a 2.5% ammonium hydroxide was prepared by diluting 1 ml of 25% ammonium hydroxide (Arcos Organics) in 9 ml of distilled water and placed in a 500 ml glass bottle. The cap was perforated to fit two tubes sealed with hot glue and parafilm. Leakage was assessed for each connecting element with two flow-meters. One fitting was used as an inlet for a pump and the second was connected to a membrane holder. The membrane holder was designed using Solidworks computer aided design (CAD) modeling software (Dassault Systems) and 3D printed on an Ender-3 printer (Creality) using poly-lactic acid (PLA) filament (Spider 3D, Israel). The printed parts were sealed with parafilm (Bemis). Both sides of the membrane holder were identical and a silicon O-ring was fitted above the membrane to isolate it. The membrane holder was connected to a second glass bottle (250 ml) through a tap. Two other taps were fitted on the second container. One was used for in-flow of air and the second was an outlet directed to a WatchGas Poli MP400 VOC sensor (WatchGas detection). The taps allowed to circulate vapors for the first container through the membrane into the second sealed container for 1 h. After 1 h, the tap above the membrane was closed and the two other taps opened allowing the headspace content to flow through to the VOC sensor. A control treatment was repeated (n=3) with a Whatman 1 filter membrane (Whatman). To assess the CNC-glycerol barrier properties, fresh solutions of CNC-glycerol were prepared as described above (5 dry wt. % of glycerol was added to a 2 wt. % solution of CNC in water, sonicated and centrifuged). Whatman 1 filters (47 mm diameter) were coated with 2 ml of the CNC-glycerol solution and let to dry. The experiment was repeated (n=3), and control and treated membranes were run in a random order. A fresh solution of ammonium hydroxide was prepared for every repeat. The airflow used in both steps of the experiment was maintained at 500 ml/min. After 1 h, the VOC sensor data were collected using the WatchGas Suite software. One point was read every 2 s for 5 min until all readings were 0.00 ppm. As depicted in FIG. 6A, in some cases, the measured VOC content exceeded the max threshold of the sensor (>200 ppm). In all three CNC treatments, the reading was consistent at 0.00 ppm. The content may have been lower than the resolution of the sensor and this is to be taken into consideration in the result analysis.

### CONCLUSION

CNC was found to reduce the blood feeding in A. Aegypti when tested on a single human hand. Broadening the investigation to an artificial feeding system and assessing the eggs laid after feeding A. Aegytpi with and without CNC confirmed that CNC can act as a chemical camouflage significantly reducing the laid eggs counted, and thus the number of blood-fed females per cage. The combined camouflage effect of CNC and the active repelling of indole further reduced egg laying and confirmed the excellent potential of CNC as a media for active repellents including commercially available ones produced in a safe and sustainable way. Extending the investigation to An. Gambiae showed reduction in egg laying when CNC was used. The biocompatibility of the CNC, the easy washing of the coating, as well as the self-assembly characteristic together with the ability for cost effective mass production, makes CNC ideal for the development of a new generation of mosquito PPE. Similar CNC formulations maybe developed to protect plants from certain insects.

To determine effectiveness of formulations of the invention, three different formulations were applied onto a human skin region. The three formulations were:
1. Formulation comprising only CNC in water [2 wt % in water];
2. Formulation comprising CNC and glycerol in water [2 wt % CNC in water and 0.1 wt % glycerol in water];
3. Formulation comprising CNC, glycerol and indole, in water [2 wt % CNC in water and 0.1 wt % glycerol in water and 1 mM].

As demonstrated in Table 1 below, a formulation comprising CNC only did not provide sufficient protection against mosquitoes. The formulation comprising CNC and glycerol and that comprising also indole, were superior.

### Coating Integrity on Skin Analysis

150 μL of three different solutions were dropped and spread on a defined 4 cm² area of the top left-hand skin region of a subject. This area was selected as an easy to observe, low hair distribution, and highly stretchable area. One repeat was performed on the back side of the left forearm, also a low-density hair area. The area was determined using the area selection tool on ImageJ.

The method used was a subjective method and was performed 5 times per treatment on different days but on the same subject.

Table 2 below provides the summary of the tests results. As may be derived from Table 2, pure CNC provided less than effective protection as it provided no stable and continuous film in almost 80% of the skin samples. The addition of glycerol dramatically increased protection as a combination thereof with CNC provided a film that was continuous and stable. That means that CNC alone, as was glycerol, is not capable of preventing semiochemicals from reaching the surface of the film. The emission of semichemical through a film formed of CNC or glycerol alone is expected to be much higher as compared with that observed for a combination of CNC and glycerol (with or without indole).

TABLE 2
a summary of the tests results.

| | | | CNC + |
|---|---|---|---|
| | | CNC + | Glycerol + |
| | CNC | Glycerol | Indole |
| | (cm²) | (cm²) | (cm²) |
| | 2.439 | 0.947 | 1.488 |
| | 2.786 | 0.63 | 0.48 |
| | 3.34 | 1.14 | 1.036 |
| | 3.48 | 0.79 | 0 |
| | 3.885 | 0 | 0.9 |
| Average (cm²) | 3.19 | 0.70 | 0.78 |
| st. dev | 0.57 | 0.44 | 0.57 |
| % exposed | 79.65 | 17.54 | 19.52 |
| surface | | | |
| st. dev percent | 14.34 | 10.88 | 14.14 |

CNC was found to reduce the blood feeding in A. Aegypti when tested on a single human hand and on an artificial feeding system by assessing the eggs laid after feeding A. Aegytpi with and without CNC and confirmed that CNC can act as a chemical camouflage significantly reducing the laid eggs counted, and thus the number of blood-fed females per cage. The combined camouflage effect of CNC and the active repelling of indole, where present, further reduced egg laying and confirmed the excellent potential of CNC as a media for active repellents produced in a safe and sustainable way. CNC's chemical barrier effect was shown in a headspace experiment where ammonium hydroxide, a known mosquito attractant and a material known to be excreted from the skin of a human subject or animal, was blocked by the CNC-glycerol coating. The biocompatibility of the CNCs, their ubiquity, as well as the self-assembly characteristic together with the ability for cost effective mass production makes CNC ideal for the development of a new generation of mosquito PPE.

1.-40. (canceled)

41. A formulation for direct application onto a skin region of a subject for reducing or preventing insects from biting the skin region and/or for reducing or preventing bite-related skin condition and/or for reducing or preventing transmittal of an insect-borne disease, the formulation comprising a skin-hydrating fluid and cellulose nanocrystals (CNC).

42. The formulation according to claim 41, wherein the reducing or preventing is achievable by minimizing, diminishing or preventing skin-semiochemicals emitted from the skin region to sufficiently disperse or spread so as to be detected by the insect.

43. The formulation according to claim 41, wherein the reducing or preventing is achievable by minimizing or reducing concentration of skin-semiochemicals emitted from the skin region to below 100 ppm or to below 100 ppb level and/or by reducing external temperature of the skin region having the formulation applied thereon.

44. The formulation according to claim 41, wherein the reducing or preventing is by forming a film of the formulation having a thickness of between 10 microns and 1 mm.

45. The formulation according to claim 41, wherein the hydrating agent is glycerol, sorbitol, propylene glycol or mixtures thereof.

46. The formulation according to claim 41, the formulation being free of an insect repellent.

47. The formulation according to claim 41, the formulation comprising indole.

48. The formulation according to claim 41, comprising CNC, glycerol and indole.

49. A formulation for minimizing, diminishing or preventing skin-semiochemicals emitted from the skin region to disperse or spread so as to be detected by a mosquito, the formulation being adapted for direct application onto an exposed skin region of a subject for forming a film of said formulation, thereby reducing or preventing mosquitos from biting the skin region, the formulation comprising glycerol, CNC and optionally indole.

50. The formulation according to claim 49, comprising indole.

51. A method for preventing or reducing insect biting, the method comprises applying an effective amount of a formulation according to claim 41 to an exposed area of a skin of a subject and forming a film or a coat of said formulation on said area of the skin.

52. The method according to any one of claim 51, wherein the biting insect is a mosquito.

53. The method according to claim 51, for preventing or minimizing or reducing a mosquito-borne disease selected from malaria, dengue, West Nile virus, chikungunya, yellow fever, dirofilariasis, filariasis, tularemia, Japanese encephalitis, Saint Louis encephalitis, Western equine encephalitis, Eastern equine encephalitis, Venezuelan equine encephalitis, Ross River fever, Barmah Forest fever, La Crosse encephalitis, Zika fever.

54. The method according to claim 51, wherein the formulation is in a form of a cosmetic formulation, or as a multipurpose formulation.

55. The method according to claim 51, wherein the formulation is in a form of a cream, a lotion, an ointment, or a spray.

56. A kit in a form of a skin applicator comprising a formulation according to claim 41 and instructions of use.
